(19) United States
(12) Patent Application Publication
(10) Pub. No.: US 2012/0033786 A1
(43) Pub. Date: Feb. 9, 2012

(54) MAMMOGRAPHY PAD

(75) Inventors: Helen Zinreich Shafer, Owings Mills, MD (US); Brian Bolton, Columbia, MD (US)

(73) Assignee: IZI MEDICAL PRODUCTS, LLC, Baltimore, MD (US)

(21) Appl. No.: 12/850,291

(22) Filed: Aug. 4, 2010

(51) Int. Cl. A61B 6/04

(52) U.S. Cl. 378/37; 378/208

(57) ABSTRACT

A mammography pad for use with a mammography unit for providing comfort to a patient during a mammography examination. The mammography pad includes a flexible substrate configured to overlie a bucky plate, a compressible material and a fastening means. The mammography pad is capable of covering a top and front surface of the compression plate, while the fastening means removably attaches a portion of the mammography pad to the mammography unit. Further, a method for administering a mammography examination with the mammography pad is disclosed by providing a mammography pad, securing the pad to the mammography unit, placing a patient's breast on the mammography pad, and compressing the patient's breast against the mammography pad.

### TECHNICAL FIELD

The present invention relates generally to medical devices, and more particularly to an apparatus and method for providing patient comfort during mammography examinations.

### BACKGROUND

During mammography examinations, a patient's breast is compressed by opposing plates attached to a mammography unit. Once under compression, an x-ray may be taken to determine the presence or absence of suspect lesions in the breast tissue. An important reason for compressing the breast is to provide a thinner cross-section of tissue for the x-rays to pass through. One of the problems with mammography is that patients may experience discomfort during compression of the breast. Although some patients can tolerate lower units of compression, higher compression units are needed to obtain a more enhanced mammogram increasing the probability of detecting cancers or suspect lesions. Unfortunately, this discomfort may lead many women to avoid mammography. While prior art devices have been designed to provide comfort for patients (i.e., gel pads, heating pads etc.), such devices provide little or no added comfort for the patient, or they lead to significant increases in the cost of examinations. Therefore, there remains a need for comfort devices to reduce or eliminate patient discomfort (i.e., discomfort caused by the coldness and pressures associated with mammography examinations), and to provide a more sanitary device thereby preventing the likelihood of an allergic reaction or cold symptoms, without adding significant costs to mammography examinations.

### BRIEF SUMMARY

An embodiment of the present invention comprises a mammography pad for use with a mammography unit for providing comfort to a patient, and aiding in the positioning of the patient's breast tissue during mammography examinations. The pad includes a substrate adapted to overlie a compression plate. The substrate has a first and second surface, and at least one score line that defines at least first portion and at least second portion of the substrate. The first portion of the second surface of the substrate includes a fastening means thereon for releasably attaching the pad to the mammography unit. The pad further includes a compressible member secured to the first surface of the substrate.

In another embodiment of the present invention, the mammography pad comprises a substrate formed from polymers, like a high density polyethylene. Additionally, the compressible member is formed from medical grade hypoallergenic materials.

In another embodiment of the present invention, the mammography pad comprise a substrate having a thickness between about 0.015 inches and 0.10 inches, a compressing member having a thickness between about 0.25 inch and 1 inch, and a fastening means having a thickness between about 0.015 inches and 0.10 inches.

In another embodiment of the present invention, the mammography pad comprise a substrate having a score line defining a first and second portion, wherein the first portion has an area equal to or less than the second portion.

In another embodiment of the present invention, the mammography pad comprise a substrate having a score line defining a first and second portion, wherein the first portion has an area equal to or greater than the second portion.

In another embodiment of the present invention, the mammography pad comprises a fastening means that is an adhesive, wherein the adhesive is a radiolucent foam tape, and includes a selectively removable liner.

In another embodiment of the present invention, the mammography pad comprises at least one wing or strap for releasably attaching the mammography pad to the compression plate, wherein the at least one strap includes a fastener at a first end of the at least one strap for releasably attaching the first end to an underside surface of the compression plate.

In another embodiment of the present invention, the mammography pad comprises radiopaque markings having origin information that may correspond to the mammography pad.

In another embodiment of the present invention, the mammography pad comprises at least one selectively removable sheet adapted to lie over the mammography pad, wherein the sheet is made from a hypoallergenic material, and optionally includes at least one tab selectively attached thereon for selectively removing the sheet from the mammography pad.

In another embodiment of the present invention, the mammography pad is adapted to be placed in a second folded position from a first planar position, wherein the first and second portions of the substrate are substantially coplanar to each other in the first position, and the first and second portions are approximately perpendicular to each other in the second position.

Another embodiment of the present invention comprises a method of administering a mammography exam using the mammography pad. The method includes providing a mammography pad adapted to overlie a compression plate of a mammography unit, wherein the mammography pad comprises a substrate having first and second surfaces, and at least one score thereby defining a first and second portion, a compressible member attached to the first surface of the substrate, and a means for fastening the pad to the compression plate. The method further includes positioning the mammography pad from a first position, wherein the first portion and the second portion are substantial coplanar to each other, to a second position wherein the first portion is approximately perpendicular to the second portion. The method further includes fastening the first portion to the compression plate. The method further includes placing a patient's breast on the mammography pad, and compressing the breast against the mammography pad.

### BRIEF DESCRIPTION OF THE FIGURES

FIG. 1 illustrates an underside perspective view of a mammography pad according to an embodiment of the subject invention;

FIG. 2a illustrate an underside view of a substrate according to an embodiment of the subject invention;

FIG. 2b illustrates a side view of the substrate according to an embodiment of the subject invention;

FIG. 3a illustrates an underside view of the mammography pad in a first position and a fastening means according to an embodiment of the subject invention;

FIG. 3b illustrates a side view of the mammography pad in the first position, the first portion being greater than the second portion, and the first portion having the fastening means according to an embodiment of the subject invention;

FIG. 4a illustrates a perspective view of the mammography pad in a second position, and including a compressible member according to an embodiment of the subject invention;

FIG. 4b illustrates a side view of the layers of the mammography pad according to an embodiment of the subject invention;

FIG. 5a illustrates a top view of a mammography pad having a compressible member and a substrate with different surface areas according to another embodiment of the subject invention;

FIG. 5b illustrates an underside view of yet another embodiment of a mammography pad having a compressible member and a substrate with different surface areas;

FIG. 6 illustrates a flow chart of an embodiment of a method of administering a mammography examination with the mammography pad described herein.

### DETAILED DESCRIPTION

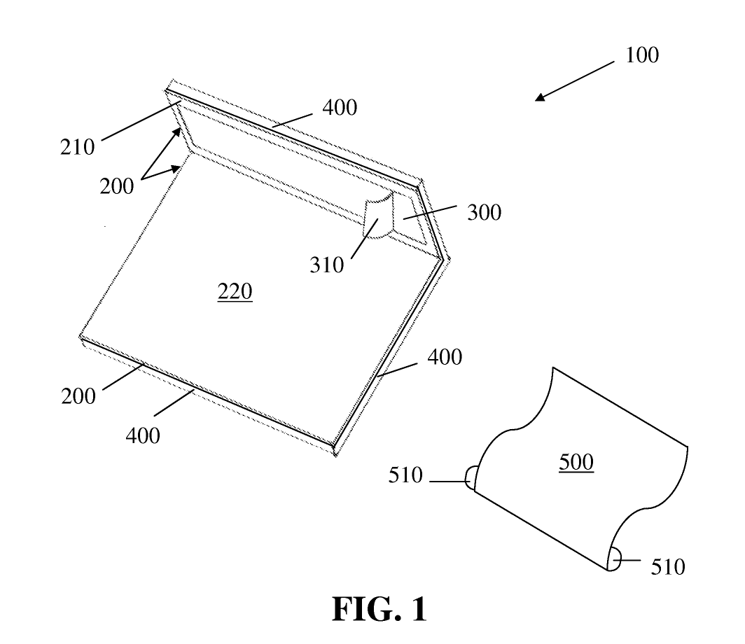

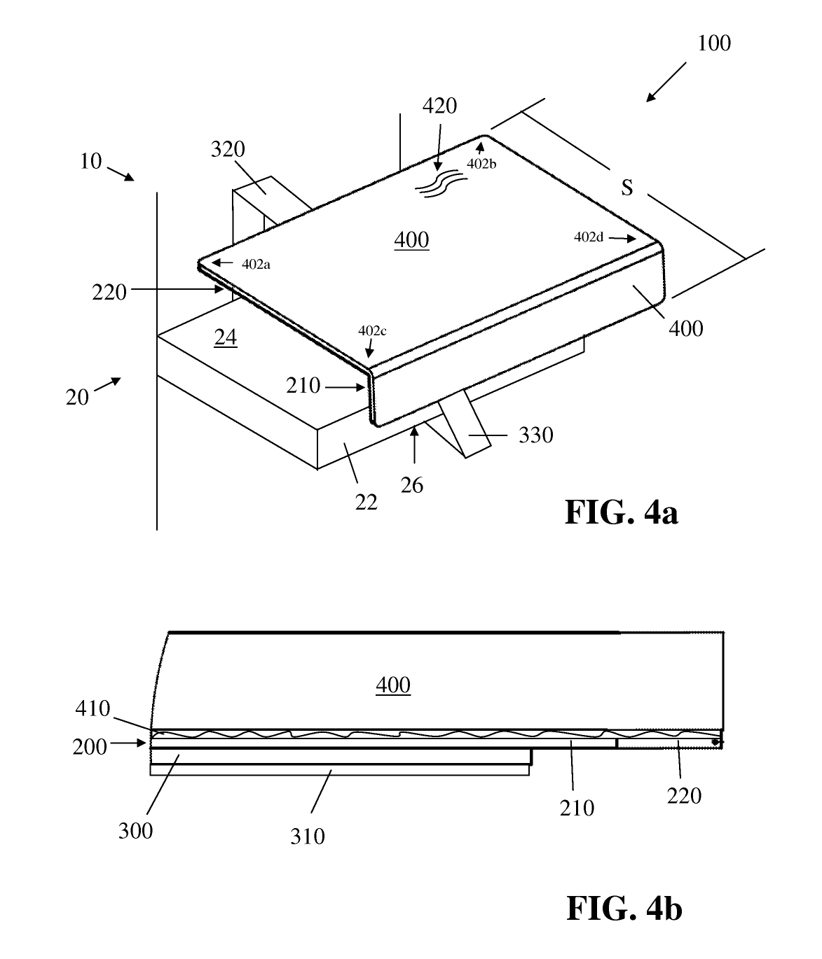

FIG. 1 illustrates an underside perspective view of a mammography pad 100 according to one embodiment of the subject invention. The mammography pad 100 may include a substrate 200, an optional fastening means 300 with a removable liner 310 for removably attaching the mammography pad 100, and a compressible member 400 for providing patient comfort. Additionally, the mammography pad 100 may further include at least one removable sheet 500 adaptable to overlie the mammography pad 100. The mammography pad 100 may be used with a mammography unit 10, as shown in FIG. 4, for providing comfort to patients during a mammography examination. In use, a mammography technician may place the mammography pad 100 on a compression plate 20, also referred to as a bucky plate, of the mammography unit 10. The technician will typically peel off the removable liner 310 from the fastening means 300 before removably attaching the mammography pad 100 to the front surface 22 of the compression plate 20 thereby preventing the mammography pad 100 from unintentionally moving during the mammography examination. The patient may then place their breast tissue on the compressible member 400, or sheet 500 of mammography pad 100, prior to the mammography unit 10 compressing the breast tissue in order to capture an image of the tissue. All the component herein may be made from a radiolucent materials.

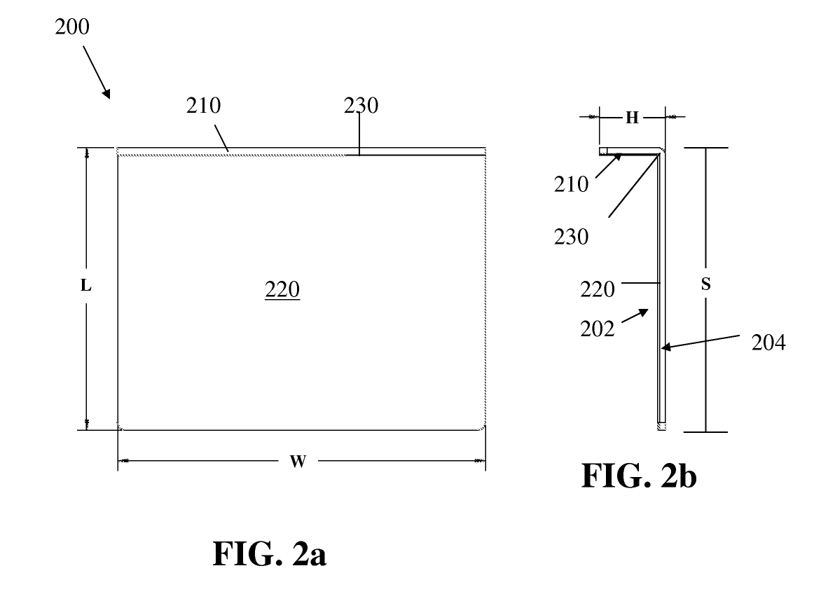

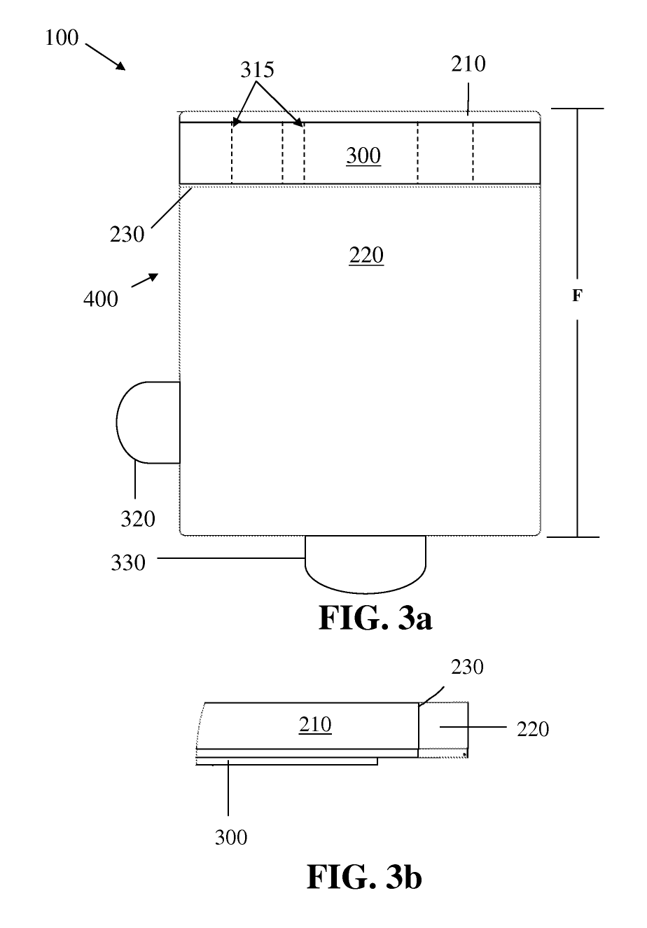

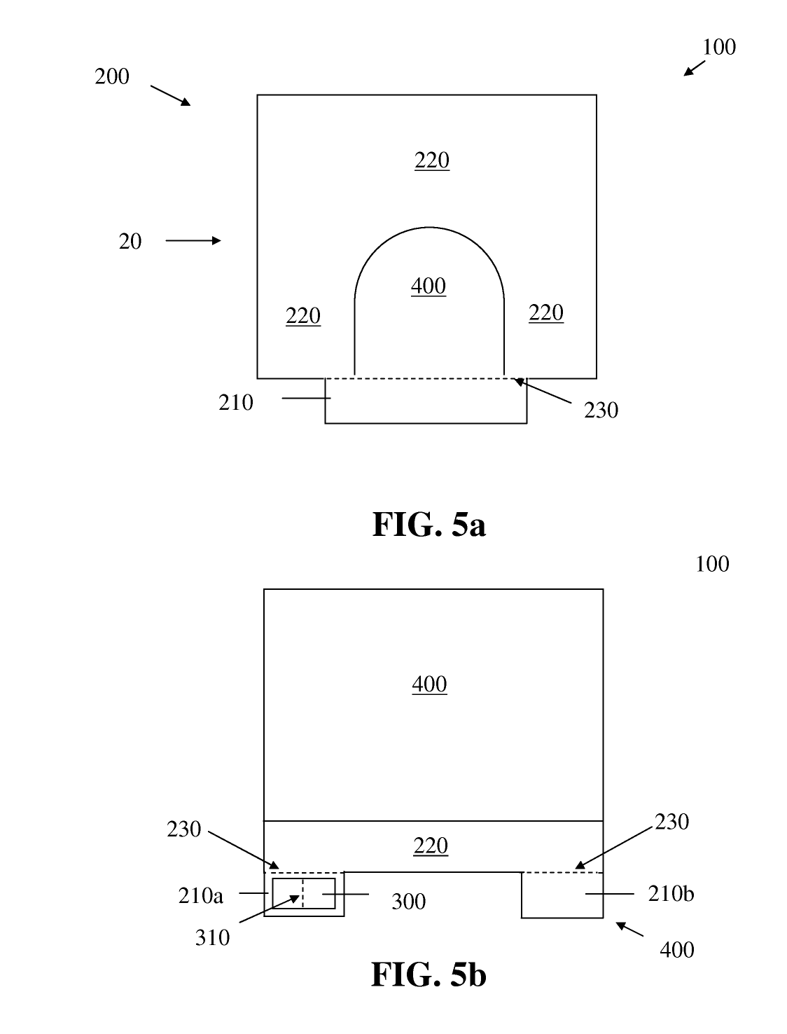

With continued reference to FIG. 1 and now FIG. 2, an underside view of the substrate 200 according to an embodiment of the subject invention is illustrated. The substrate 200 may be made of a radiolucent material. Additionally, the substrate 200 may be made of a polymer, or any suitable material capable of adapting to surfaces of a compression plate 20 of a mammography unit 10 known to a person having ordinary skill in the art. The polymer may be rigid or flexible. In one embodiment, the polymer may be a high density polyethylene (e.g., Hitec®). The substrate 200 has a first surface 202 and a second surface 204. The surfaces of the substrate 200 may be substantially smooth and capable of allowing for movement of the substrate 200 across the surface of the compression plate 20 for positioning the mammography pad. 100. The first surface 202 may be scored thereby creating at least one scoreline or score 230 for partitioning the substrate 200 and thereby defining a first portion 210 and a second portion 220 of the substrate 200. For purposes consistent with the invention disclosed herein, scoring means to facilitate the bending of the substrate 200 thereby creating an angle. The score 230 may facilitate folding the substrate 200 from a first planar position F (as shown in FIG. 3a), wherein the first portion 210 and the second portion 220 may be substantially coplanar, meaning substantially existing on the same plane, to one another, to a second folded position S (as shown if FIG. 2b), wherein the first portion 210 is approximately perpendicular to the second portion 220, and the first portion 210 and the second portion 220 share a common edge. The score 230 may also extend the full width W of the substrate 200. In another embodiment, a plurality of scores 230, or serrations, as shown in FIG. 5a, may be used as a means for releasably attaching the substrate 200 to the mammography unit 10 by facilitating the folding of the mammography pad 100. In this embodiment, the plurality of scores 230 may be a similar size to each other and spaced apart from each other thereby defining separate portions on the substrate 200. The spacing may be random, or an equidistance apart. Additionally, a technician or user, may fold or crease the substrate 200 without a score line, with or without a tool, or the substrate 200 may be self-scoring in order to divide the substrate 200 into portions. For example, a laser etch may be used to create the score 230, thereby defining the first 210 and second 220 portions, for configuring the substrate 200 to bend from the first position F, to the second position S. In one embodiment, the first portion 210 may have an area less than or equal to that of the second portion 220. For example, the first portion 210 may have a height H, as shown in FIG. 2b, of about 2.5 inches. In another embodiment, as shown in FIG. 3b, the first portion 210 may have an area greater than that of the second portion 220. As shown in FIG. 2b, the substrate 200 is in the second position S, where the area of the first portion 210 is less that the area of the second portion 220. The first portion 210 may be of a size similar to the front surface 22 of the compression plate 20 of the mammography unit 10. Additionally, the substrate 200 may have a thickness of any size such that it will not negatively affect the imaging procedure. In one embodiment, the thickness of the substrate 200 may be between about 0.015 inches and 0.50 inches. In certain embodiments it is contemplated as being approximately 0.030 inches. In a further embodiment it is contemplated as being approximately 0.10 inches.

In another embodiment, the substrate 200 may be preformed in an substantially folded shape thereby eliminating the need for scoring the substrate 200 for later folding. In this embodiment, the mammography pad 100 may be placed on the compression plate 20 for providing patient comfort as described herein.

FIG. 3a illustrates an underside view of the mammography pad 100 in the first position F, and having a first portion 210 with an area less that the second portion 220 and a fastening means 300 removably secured to the first portion 210 for removably attaching the substrate 200 to the compression plate 20 (FIG. 4). The fastening means 300 may be an adhesive, a medical grade foam tape, a double-sided foam tape like Remo-one®, hook and loop, or any fastener capable of removably attaching the substrate 200 to a surface of a mammography unit known to a person having ordinary skill in the art. Hook and loop may be desirable when the mammography pad 100 remains in place for multiple uses and the sheet 500 (FIG. 1) is changed with each patient or examination. In this embodiment, the mammography pad 100 and the compression plate may both include corresponding hook and loop fasteners 300 secured thereon, and the mammography pad 100 remains selectively attached to the mammography unit 10 while only the sheet 500 is changed. In addition to hook and loop, screws, bolts, snaps or the like may be used to removably attach the mammography pad 100 to the mammography unit 10. Again this configuration may be useful when the mammography pad 100 remains in place relative to the compression plate 20 and only the sheets 500 are changed between procedures. The fastening means 300 may be removably secured to the first portion 210 of the substrate 200. The fastening means 300 may be positioned in the center of the first portion 210 or at any other position on the first portion 210 chosen with sound judgment. The fastening means 300 may be easily removed from the compression plate 20 with minimal effort. Additionally, the fastening means 300 in the form of an adhesive layer may have a thickness of any size such that it will not negatively affect the imaging procedure. In one embodiment, the thickness of the fastening means 300 may be between about 0.015 inches and 0.10 inches, and in another embodiment about 0.031 inches thick. This fastening means 300 may further include a removable liner 310 attached thereon. The removable liner 310 may remain removably attached to the fastening means 300 prior to use of the mammography pad 100 to eliminate the possibility of the fastening means 300 attaching to an unintended surface. Alternatively the removable liner 310 may include at least one tear line 315 as shown in FIG. 3a. The tear line 315 allows for individual sections of the removable liner 310 to be removed thereby partially exposing any one portion or specific portions of the fastening means 300. In operation, a technician or similar person may remove the liner 310 or sections thereof in preparation for removably attaching the mammography pad 100 to the compression plate 20. Additionally, the removable liner 310 having a plurality of tear lines 315 may allow for single or multiple use of the same mammography pad 100. If the liner 310 includes a plurality of tear lines 315, the tear lines 315 may have different sizes or shapes, or may be spaced apart uniformly or in a non-uniform manner. The liner 310 may be removed prior to forming the mammography pad 100 from the first position F to the second position S, or after forming the mammography pad 100 into the second position S. In an embodiment where the mammography pad 100 has a substrate 200 preformed into a substantially folded position, the liner 310 may be removed prior to placing the mammography pad 100 on the compression plate 20 regardless of any positions. Once the liner 310 is removed, the first portion 210 may be removably attached to the mammography unit 10, more specifically the front surface 22 of the compression plate 20. The liner 310 and fastening means 300 may radiolucent. Alternatively, a technician may elect to use the mammography pad 100 without removing the liner 310, or using the mammography pad 100 without the optional fastening means 300. In this embodiment, the patient's person may create enough friction or resistance to position the mammography pad 100 on the compression plate 20. Additionally, in one embodiment, the fastening means 300 is contemplated as being readily releasable such that the mammography pad 100 may be repositioned between imaging of the same patient, or between examinations for the same patient. For example, a patient may begin her first examination using the mammography pad 100 on one day, and later return on a subsequent day for a second examination using the same pad. Therefore, the fastening means 300 may be used for a single or multiple applications for releasably attaching the mammography pad 100 to the mammography unit 10.

With continued reference to FIG. 3a, in one embodiment the mammography pad 100 may include a wing or first strap 320 selectively attached to the mammography pad 100 for releasably attaching the mammography pad 100 to the compression plate 20. The first strap 320 may be attached to any side of the substrate 200 or in the alternative to the compressible member 400. In one embodiment, the first strap 320 is attached to the second portion 220 of the substrate 200. In another embodiment, the mammography pad 100 may further include a second strap 330. The second strap 330 may be attached to the first portion 210 such that the first strap 320, and the second strap 330 may attach under the compression plate 20, as shown in FIG. 4a. The first strap 320 may be used together with the fastening means 300. In this embodiment, the first strap 320 provides an addition means for attaching the mammography pad 100 to the compression plate 20. The straps may be made from any suitable material capable of releasably attaching the mammography pad 100 to the compression plate 20 known to a person having ordinary skill in the art. Additionally, the first strap 320 or second strap 330 may be made from a radiolucent material. In one embodiment, the first strap 320 may includes a fastener (not shown) for attaching the first strap 320 to an underside surface 26 (FIG. 4a) of the compression plate 20. Where there are two or more straps attached to the mammography pad, the additional straps may also include fasteners for attaching the straps to each other under the compression plate 20, or for attaching the straps to the underside surface 26. The fastener may be made from the same materials as the fastening means 300, or any material capable of attaching the straps to the compression plate 20 known to a person having ordinary skill in the art.

In one embodiment, the first strap 320 and second strap 330 may be selectively attached the mammography pad 100 such that the first strap 320 and second strap 330 is approximately perpendicular to the fastening means 300. In another embodiment, the first strap 320 and second strap 330 may extend along different sides of the compression plate 20 such that the first strap 320 and second strap 330 are approximately perpendicular to each other. Additionally, the first strap 320 and second strap 330 may extend along opposed sides of the compression plate 20 such that the first strap 320 and second strap 330 are approximately parallel to one another.

In another embodiment, the first strap 320 may be made from an elastic material such that the first strap 320 may be stretched to fit over the compression plate 20 causing elastic forces for releasably attaching the mammography pad 100 to the compression plate 20. Additionally, the straps may be used with an embodiment of the mammography pad 100 that does not include the fastening means 300.

FIG. 4a illustrates a perspective view of the mammography pad 100 in the second position S with the compressible member 400 secured to surface of the substrate 200. The compressible member 400 may be formed from a foam, a medical grade foam, a hypoallergenic foam, or any material capable of compressing when used with a mammography unit 10 known to a person having ordinary skill in the art. Hypoallergenic materials are materials that have a lesser likelihood, if any, to cause an allergic reaction. The compressible member 400 may have a thickness of any size such that it will not negatively affect the imaging procedure. In one embodiment, the thickness of the compressible member 400 may be between about 0.25 inches and 1 inch. In certain embodiments the compressible member may have thicknesses of about 0.375 inches or about 0.5 inches. The compressible member 400 may be radiolucent, leaving no artifacts on any readable media. However, for example, when using a mammography phantom, the mammography pad 100 may alter grayscale values (on a 256 grayscale) by no more than 10 values. With continued reference to FIG. 4a and now FIG. 4b, the compressible member 400 may be permanently secured to the substrate 200 using a securing means 410. The securing means 410 may be an adhesive, like a foam tape, more particularly a medical grade foam tape. The securing means 410 for attaching the substrate 200 to the compressible member 400 may differ from the fastening means 300, in that the securing means 410 may permanently secure substrate 200 to the compressible member 400, whereas the fastening means 300 may be removable. In another embodiment, the compressible member 400 may be secured to the substrate 200 using a liquid adhesive, or any other element capable of securing the compressible member 400 to the substrate 200 known to a person having ordinary skill in the art. The adhesive may be used on the entire area of the compressible member 400, or near the outer edges (402 a-d) of the compressible member 400, thereby securing the compressible member 400 to the substrate 200. Alternatively, the adhesive may be used in a single area, or selective areas on the compressible member 400 or the substrate 200. The area may be around or about the centermost area of the compressible member 400, or in selective areas chosen with sound judgment, and capable of securing the compressible member 400 to the substrate 200. The adhesive may be applied to the compressible member 400, the substrate 200 or both for securing the components. For example, a double sided foam tape may be used to secure both components together. Here the adhesive surface may be attached to both the substrate 200 and the compressible member 400. Additionally, the compressible member 400 may be secured to the substrate 200 after the substrate 200 is scored and when the substrate 200 is in the first position F. However, the compressible member 400 may be secured while the substrate 200 is in the second position S, or in a preformed substantially folded position without exceeding the scope of the subject invention. It shall appreciated that the compressible member 400 may be used with any of the embodiments of the substrate 200 disclosed herein.

With continued reference to FIG. 4, and now FIG. 5, in another embodiment, the compressible member 400 may have a thickness equal to or greater than the substrate 200 thereby providing a higher compressibility, however a compressible member 400 having a thickness less than the substrate 200 may suffice for providing comfort to the patient. Additionally, the surface area of the substrate 200 may be different than the surface area of the compressible member 400. For example, as shown in FIG. 5a, the compressible member 400 may have a surface area comparable to the size of a patients breast tissue, while the surface area of the substrate 200 covers the entire surface of the compression plate 20. In the alternative, the surface area of the compressible member 400 may be larger than the surface area of the substrate 200. Here, as shown in FIG. 5b, the surface area of the compressible member 400 may cover the top surface 24 and the front surface 22 of the compression plate 20, while the surface area of the substrate 200 covers the front surface 22 and at least a portion of the top surface 24 of the compression plate 20. Alternatively, the substrate 200 may include at least two first portions 210a and 210b, as shown in FIG. 5b, for removably attaching the compressible member 400 to the compression plate 20. In this embodiment, the fastening means 300 may be included on one of the portions or both.

In yet another embodiment, the substrate 200 may form a frame or place holder (not shown) along the boundary of the compressible member 400, thereby leaving the center area of the compressible member resting on the top surface 24.

In another embodiment, with continued reference to FIG. 1, the removable sheet 500 may be adapted to overlie at least a portion of the mammography pad 100. For example, the removable sheet 500 may cover only portions of the mammography pad 100 where the patient will come in contact, or the removable sheet 500 may cover the entire mammography pad 100. The removable sheet 500 may be used in lieu of changing the mammography pad 100 after each use, a technician or patient may elect to have the sheet 500 change. The sheet 500 may include a tab 510 for assisting with removing the sheet 500 from the mammography pad 100. Additionally, the sheet 500 may be made of the same materials (i.e. hypoallergenic, medical grade) as the compressible member 400, or any material capable of resisting moisture and being radiolucent.

With continued reference to FIG. 1 and FIG. 4, in yet a further embodiment, the substrate 200 may include radiopaque markings 420 having information corresponding to the origin of the mammography pad 100. For example, the radiopaque markings 420 may include manufacturing or distribution location information. The information may include information about the manufacturer, any covered intellectual property, or information regarding the materials that make up the mammography pad 100. Alternatively, the substrate 200 or the fastening means 300 may include radiopaque markings 420 for imparting information on any readable media. Additionally, for breast cancer awareness, the compressible member 400 may be a pink color. The compressible member 400 may have additional colors or patterns, or a combination of colors and patterns (i.e., red, white, blue) or transparent, based on a person's preference.

In yet another embodiment, the substrate 200 may include a second score (not shown). In this embodiment, the mammography pad 100 may include three portions for fitting over three surfaces of the compression plate 20. For example, the substrate 200 may cover the top surface 24, the front surface 22, and the underside surface 26 of the compression plate 20. Here the compressible member 400 may also extend over the substrate 200, which extends over the three surfaces, or the compressible member 400 may only extend over the surfaces the patient may come in contact with, like the front surface 22, the top surface 24 or both.

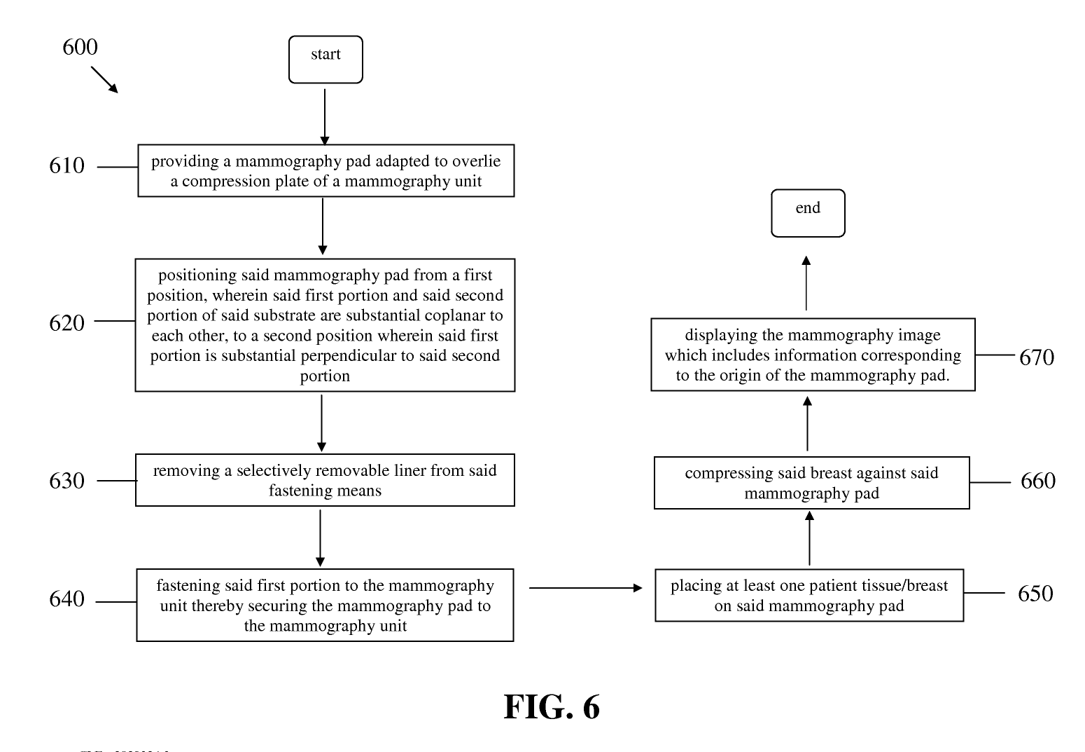

FIG. 6 illustrates a flow chart of an embodiment of a method 600 for administering a mammography examination using the mammography pad 100 described herein. In step 610, providing the mammography pad 100 that may be adapted to overlie a compression plate 20 of a mammography unit 10. In step 620, positioning the mammography pad 100 from the first position F to the second position S. In step 630, removing the selectively removable liner 310 from the fastening means 300. It is appreciated that step 630 may be performed prior to step 620. Additionally, if liner 310 is not present on fastening means 300, the person may directly proceed to step 640 following step 620. In step 640, fastening said first portion F to the mammography unit 10 thereby removably attaching the mammography pad 100 to the mammography unit 10. For example, a technician may simply apply pressure to the portion of the mammography pad 100 where the fastening means 300 is positioned, thereby fastening it to a surface of the mammography unit 10. In step 650, placing at least one patient breast tissue on the mammography pad 100. In step 660, compressing the at least one breast against the mammography pad 100, thereby providing comfort to the patient during the examination. In step 670, capturing and displaying the mammography image, which may include information corresponding to the origin of the mammography pad 100.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A mammography pad used in connection with a mammography unit compression plate, the pad comprising:
a substrate adapted to overlie the compression plate, said substrate having a first surface and a second surfaces;
wherein said substrate further includes at least one score thereby defining a first portion adapted to overlie at least a portion of a front surface of the compression plate, and a second portion adapted to overlie at least a portion of a top surface of the compression plate,
wherein said first portion includes a fastening means attached thereon; and
a compressible member having a surface attached to at least a portion of said first surface of said substrate.

2. The mammography pad of claim 1 wherein said substrate is formed from polymers.

3. The mammography pad of claim 1 wherein said substrate has a thickness between about 0.015 inches and 0.10 inches.

4. The mammography pad of claim 1 wherein said first and second portions are substantially coplanar to each other when said substrate is in a first planar position, and said first and second portions form an angle when said substrate is in a second folded position.

5. The mammography pad of claim 1 wherein said fastening means is an adhesive.

6. The mammography pad of claim 5 wherein said adhesive is a double-sided foam tape.

7. The mammography pad of claim 1 wherein said fastening means has a thickness between about 0.015 inches and 0.10 inches.

8. The mammography pad of claim 1 wherein the compressible member is a foam.

9. The mammography pad of claim 8 wherein said foam is substantially hypoallergenic.

10. The mammography pad of claim 1 wherein said compressing member has a thickness between about 0.25 inch and 1 inch.

11. The mammography pad of claim 2 wherein said polymer is a polyethylene.

12. The mammography pad of claim 1 further comprising at least one radiopaque marking having origin information corresponding to the mammography pad.

13. The mammography pad of claim 1 further comprising at least one selectively removable sheet adapted to overlie the mammography pad.

14. The mammography pad of claim 13 wherein said sheet is radiolucent.

15. The mammography pad of claim 13 wherein said sheet includes at least one tab selectively attached thereon for selectively removing said sheet from the mammography pad.

16. A method for providing patient comfort during a mammography examination comprising the steps of:
providing a mammography pad adapted to overlie a compression plate of a mammography unit, wherein said mammography pad comprises:
a substrate having first and second surfaces, and at least one score thereby defining a first and second portions;
a compressible member attached to said first surface of said substrate; and
a fastening means;
attaching said mammography pad to the mammography unit;
placing a patient's at least one breast on said mammography pad; and
compressing said at least one breast against said mammography pad.

17. The method of claim 16 wherein the step of attaching said mammography pad to the mammography unit further includes:
positioning said mammography pad from a first planar position, wherein said first portion and said second portion of said substrate are substantial coplanar to each other, to a second folded position wherein said first portion and said second portion form an angle therebetween;
removing a selectively removable liner from said fastening means; and
attaching said first portion to the mammography unit.

18. The method of claim 16 wherein said substrate is formed from polymers.

19. The method of claim 16 wherein said compressible member is substantially hypoallergenic.

20. A mammography pad used in connection with a mammography unit compression plate, the pad comprising:
a substrate having a first portion and a second portion, wherein said second portion is adapted to overlie at least a portion of a top surface of the compression plate, and said first portion is approximately perpendicular to said second portion; and
a compressible member having a surface attached to at least a portion of said substrate.

21. The mammography pad of claim 20 further comprising a fastening means attached to said front portion for releasably attaching said substrate to the compression plate.

22. The mammography pad of claim 21, wherein said fastening means is an adhesive.
